(19) United States
(12) Patent Application Publication
(10) Pub. No.: US 2018/0161544 A1
(43) Pub. Date: Jun. 14, 2018

(54) MEDICAL ARTICLE SAFETY CAGE

(71) Applicant: Steven F. Bierman, Del Mar, CA (US)

(72) Inventor: Steven F. Bierman, Del Mar, CA (US)

(21) Appl. No.: 15/894,792

(22) Filed: Feb. 12, 2018

Related U.S. Application Data
(63) Continuation-in-part of application No. PCT/US2016/046388, filed on Aug. 10, 2016.
(60) Provisional application No. 62/204,751, filed on Aug. 13, 2015.

(51) Int. Cl. A61M 25/02; A61M 25/00

(52) U.S. Cl. CPC A61M 25/02; A61M 25/0097; A61M 2025/028; A61M 2025/0266; A61M 2025/0246

(57) ABSTRACT

A cage or enclosure is provided for irreversibly locking around at least a portion of a catheter hub or other medical article to inhibit access to a lumen through the catheter hub. The cage includes a base, a cover, and a latching mechanism. The cage can further include a hinge. When open, a receptacle formed by the cage is accessible for receiving the catheter hub. When closed, the latching mechanism prevents the cage from being opened unless the latching member is cut-off the cage or the cage is visibly damaged. The cage could be used on a variety of medical articles including: a needleless valve or connector; an overlying cap on top of the valve or connector; administration tubing; a side port or connector on the side port; and any overlying cap on the side port or connector.

### CROSS-REFERENCE TO RELATED APPLICATION INFORMATION

This application claims the benefit under 35 U.S.C. § 120 and 35 U.S.C. § 365(c) as a continuation-in-part of International Application No. PCT/US2016/046388, filed Aug. 10, 2016, which claims priority to and the benefit of Provisional Application No. 62/204,751, filed Aug. 13, 2015, the entirety of each of which is hereby incorporated by reference herein and should be considered a part of this specification.

### BACKGROUND

### Field

The present disclosure is generally directed to containers for encapsulating articles, and more particularly, to a safety device for inhibiting access to a lumen in a medical article (such as, for example, a catheter, side port of I.V. bag, etc.) by encapsulating the medical article.

### Description of the Related Art

Caps have been used by healthcare providers to cover ports into medical articles when the medical article is in flow communication with a patient's vasculature or bloodstream. The cap maintains a clean environment at an entrance to the lumen in flow communication with the patient's vasculature. Once the cap is removed, the healthcare provider is able to inject medication into the patient via the lumen through the medical article. In certain cases, the medical article may be disposed for days or months during medical treatment. Patient's may be in the hospital the entire duration of treatment or may spend time away from the hospital during treatment while returning to the hospital for periodic checkups.

During the time a patient is unsupervised by healthcare providers, the patient could self-medicate by injecting a drug into their bloodstream against doctor's instructions. Acquiring a needle and taking the steps associated with using the needle to access the bloodstream presents a natural barrier for a patient to self-medicate. However, if the patient has a catheter or I.V. bag already in flow communication with their bloodstream, the catheter or bag may include a port which simplifies drug injection by the patient.

Various medical devices, for example, catheters, cannulas, sheaths, etc., are often introduced into a patient, for example, in an artery, vein, body cavity, or drainage site, to deliver fluids to or withdraw fluids from the patient. For example, an intravenous catheter can be introduced into a patient's blood vessel and be left in the blood vessel for an extended period of time. When the intravenous catheter is not being used by the healthcare provider, the healthcare provider may secure a removable cap or other structure over the catheter lumen to maintain cleanliness. The cap or other structure, however, will not deter the patient from injecting drugs through the catheter lumen if desired by the patient.

### SUMMARY

The cage described herein advantageously provides a healthcare provider the ability to inhibit the patient from surreptitious injecting drugs through a medical article enclosed within the cage. Thus, the cage provides a visual indication if the patient tampers with the cage.

In certain embodiments, a medical safety device is provided which is configured to irreversibly lock about at least a portion of a medical article. The device includes a base and a cover configured to be positioned on the base to form a cage. The cage is sized to enclose the medical article. The device includes a latching mechanism configured to irreversibly lock the cover to the base so as to inhibit access to a lumen in the medical article.

In certain embodiments, a medical safety device is provided which is configured to inhibit access to at least a portion of a medical article. The device includes a base and a cover. The cover is movable relative to the base between a first position and a second position. The base and the cover are configured to receive the portion of the medical article when in the first position and to enclose the portion of the medical article when in the second position. The medical safety device further includes a latching mechanism configured to secure the cover to the base so as to prevent the portion of the medical article from being removed from the base and the cover when the base and the cover are in the second position.

In certain embodiments, a method for inhibiting access to at least a portion of a medical article is disclosed. The method includes providing a cage comprising a first portion and a second portion, the second portion being movable relative to the first portion, disposing the portion of the medical article in the cage, and positioning the first portion relative to the second portion to enclose the portion of the medical article in the cage. The method further includes latching the first portion to the second portion so as to inhibit at least the portion of the medical article from being removed from the cage.

### BRIEF DESCRIPTION OF THE DRAWINGS

The foregoing and other features, aspects, and advantages of the embodiments of the invention are described in detail below with reference to the drawings of various embodiments, which are intended to illustrate and not to limit the embodiments of the invention. The drawings comprise the following figures in which:

FIG. 1 is a perspective view of an embodiment of a catheter assembly inserted in a patient so that a lumen of the catheter assembly is in flow communication with a bloodstream of the patient. The assembly includes a catheter hub, a needleless valve, and a cap covering an end of the valve.

FIG. 2 is similar to FIG. 1 except a cage according to a preferred embodiment of the present invention is locked around the catheter assembly inhibiting the patient from accessing the cap and the lumen therein.

FIG. 3 is a plane view of the cage from FIG. 2 in an open and unlocked condition with the catheter assembly shown in dashed lines within a receptacle formed by the cage.

FIG. 4 illustrates another embodiment of the cage from FIG. 2 configured to lock around a side port of an IV bag to inhibit the patient from accessing the side port.

FIG. 5 illustrates an embodiment of a sheath configured to enclose a catheter assembly (similar to FIG. 1) to inhibit the patient from accessing the port.

FIG. 6 illustrates the sheath illustrated in FIG. 5 in a closed condition with the catheter assembly within the sheath.

### DETAILED DESCRIPTION

To assist in the description of the components of the cage, the following coordinate terms are used. A “longitudinal axis” is generally parallel to the medical article in the cage. A “lateral axis” is normal to the longitudinal axis. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. The terms “proximal” and “distal” are used in reference to the center of the patient's body, as will be understood by one of skill in the art. The terms “upper,” “lower,” “top,” “bottom,” “underside,” “upperside” and the like, which are used to describe the present safety device, are used in reference to the illustrated orientation of the embodiments.

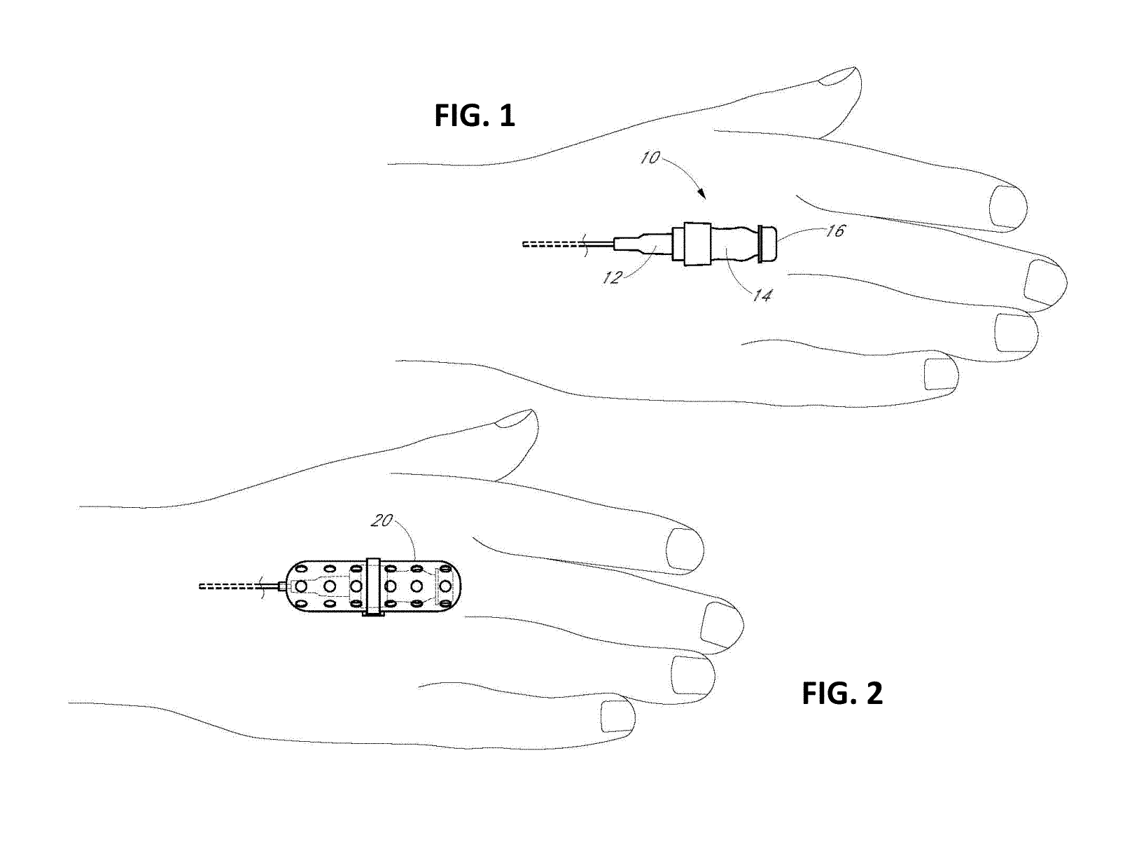

FIG. 1 is a perspective view of an embodiment of a catheter assembly 10 inserted in a patient. The catheter assembly 10 includes a catheter hub 12, a needleless valve 14, and a cap 16 covering an end of the valve 14. Referring to FIGS. 1-3, a cage 20 includes a base 22 and a cover 24 positionable over the base 22 to close the cage 20 around the catheter assembly 10.

The present embodiment of the cage 20 is disclosed in the context of placing an exemplary multi-piece, tubular catheter assembly 10 inside the cage 20. However, the principles of the present invention are not limited to the placement of a multi-piece, tubular catheter assembly 10. Instead, it will be understood in light of the present disclosure that the cage 20 disclosed herein also can be successfully utilized in connection with placing one or more other types of medical articles, including other types of catheters, fluid drainage and delivery tubes, and single or multi-lumen catheters in the cage 20. For example, FIG. 4 illustrates another embodiment of the cage 20 illustrated in FIG. 2 that is configured to lock around a side port 46 of an IV bag 48 to inhibit the patient from accessing the side port 46. Thus, the cage 20 disclosed herein can also be configured to receive central venous catheters, peripherally inserted central catheters, hemodialysis catheters, surgical drainage tubes, tear-away sheaths, multi-piece sheaths, PICC lines, IV lines, scopes, needleless valves or connectors, an overlying cap on top of a valve or connector, administration tubing, a side port or connector on the side port, any overlying cap on the side port or connector, as well as electrical conduit for wires or cables connected to external or implanted electronic devices or sensors. As explained above, the medical articles listed above may be placed within the cage 20 to inhibit the patient from accessing the medical article to self-medicate. One skilled in the art can also find additional applications for the devices and systems disclosed herein. Thus, the illustration and description of the cage 20 in connection with a catheter assembly 10 is merely exemplary of one possible application of the cage 20.

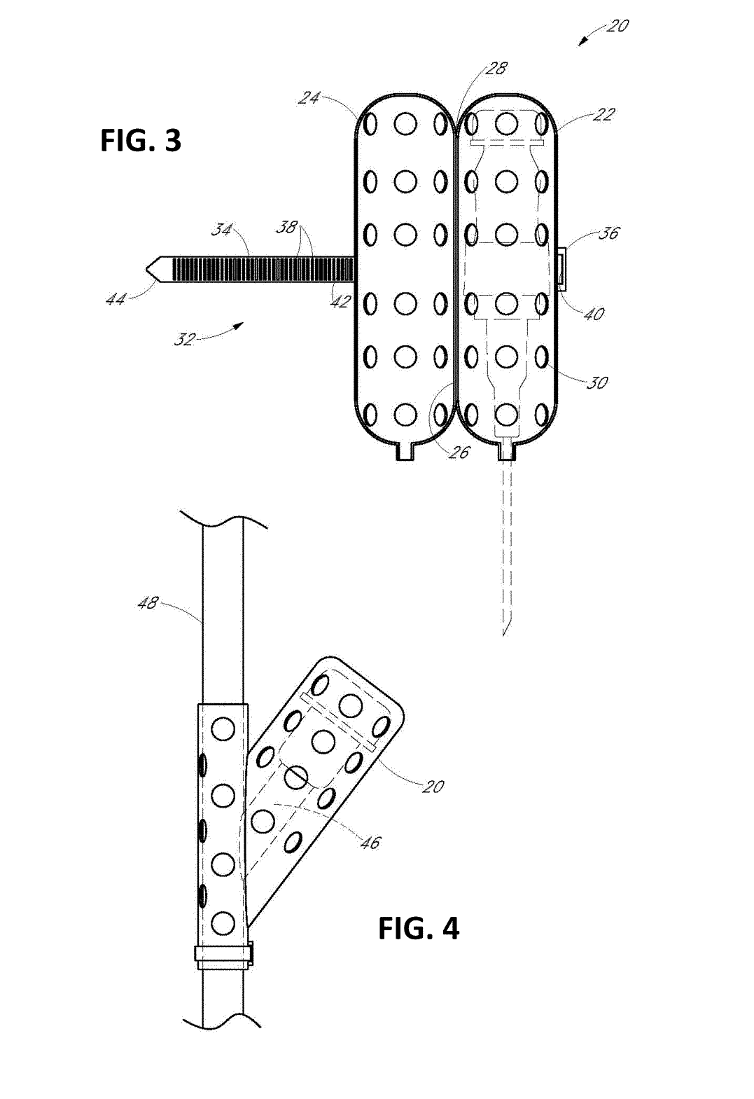

In the embodiment illustrated in FIGS. 2 and 3, each of the base 22 and the cover 24 have an elongated oval shape and together form a receptacle for the catheter assembly 10. The base 22 and the cover 24, however, can be configured in a wide variety of shapes as well, such as circular, square, triangular or the like in order to suit a particular application. The base 22 and the cover 24 together define an enclosure, cage, or container 20 into which the catheter assembly 10 or other medical article can be placed and secured. The shape of the cage 20 preferably is selected so that the catheter assembly 10 cannot be removed from the cage 20 without opening the cage 20. Thus, any openings in the cage 20 to accommodate a medical line (e.g., a catheter shaft and/or tubing) when the cage 20 is in the closed condition are sized to prevent the catheter assembly 10 from also passing through the opening. For example, the opening in the cage 20 for the catheter shaft illustrated in FIG. 2 is sized to prevent the catheter hub 12 from passing through the opening if the catheter shaft were pulled by the patient. Also, a lateral dimension of the cage 20 desirably allows the healthcare provider to easily and naturally grip the cage 20 to manipulate the cage 20.

Although the cage 20 incorporating the principles of this invention may be formed in various shapes and sizes, the preferred embodiment for use with a tubular catheter assembly 10 has the general form of an elongated oval shape with a circular or semi-circular cross-section. The length of the cage 20 is approximately 1-3 inches while the diameter is approximately ½-1 inch. The above dimensions allow many of the more common catheter assemblies 10 to be received within the cage 20. Alternative dimensions may also be used to house alternate sized catheter assemblies 10.

In the illustrated embodiment, the cover 24 is attached to the base 22 (FIG. 3) at a portion of an edge 26 of the base 22 by a living or a piano type hinge 28. In the illustrated embodiment, the edge 26 of the base 22 extends about a perimeter of the base 22 at locations where the base 22 contacts the cover 24 when the cage 20 is in the closed condition. The hinge 28 in this embodiment is integral with the base 22 and the cover 24, although a separate hinge may be provided or even no hinge. Thus, although the cage 20 in the illustrated embodiment has its cover 24 hingedly connected to the base 22, in an alternative embodiment, the hinged connection could be eliminated entirely and the cover 24 could be entirely separable from the base 22.

In certain embodiments, the base 22 includes a recess or channel along the edge 26 for engaging with an edge or lip of the cover 24 when the cage 20 is in the closed condition. In such an embodiment, the cage 20 need not include the hinge 28 since the engagement between the edge 26 of the base 22 and the lip of the cover 24 will maintain at least relative lateral and longitudinal positions of the cover 24 over the base 22. When the cage 20 is closed as shown in FIG. 2, the edge 26 of the base 22 opposes an edge of the cover 24.

The receptacle defined by the base 22 and the cover 24 is capable of receiving a portion or length of the catheter assembly 10 or other medical article and is generally configured to house or enclose the catheter assembly 10. In the illustrated embodiment an inner surface contour of the cage 20 preferably is selected depending on the geometry of the portion of the catheter assembly 10 to be retained. For example, in a cage 20 that is configured to retain a portion of a catheter assembly 10 that has a constant outer diameter, the receptacle formed by the cage 20 preferably has a constant radius along its length between the top and bottom ends of the cage 20. In contrast, in a cage 20 configured to retain a portion of a medical article that has a contoured outer surface or Y-shape, the receptacle formed by the cage 20 may have a matching inner surface. Of course the receptacle formed by the cage 20 is not so limited and can comprise a plurality of different radii and/or tapering regions. In this way, the size and shape of the cage 20 can be chosen to match or to approximate the size and shape of the medical article 10 or portion thereof to be covered.

In certain embodiments, the catheter assembly 10 is secured within the cage 20 by the engagement of one or more abutment surfaces of the catheter assembly 10 with one or more contact surfaces of the cage 20. These abutment surfaces may include, for example, a surface on the top end of the catheter assembly 10 and a surface on the bottom end of the catheter assembly 10. Each abutment surface can cooperate with a contact surface within the cage 20 to inhibit movement of the catheter assembly 10 once in the cage 20. For example, in the illustrated embodiment, the surface on the inside bottom of the cage 20 would limit longitudinal movement of the catheter assembly 10 in one direction relative to the cage 20 when the surface on the bottom of the catheter assembly 10 contacts the surface on the inside bottom of the cage 20.

One of the base 20 or the cover 22 can extend through an arc of greater than 180° about a longitudinal axis to form the cage 20, so as to provide a degree of snap-fit between the catheter assembly 10 and the cage 20. To facilitate placement of the catheter assembly 10 in the base 22 of the cage 20, the base 22 can include one or more features, such as an internal wall or post to guide the catheter assembly 10 into the base 22. In certain embodiments, the base 22 and the cover 24 each extend through an arc of 180° forming a clamshell shape. In the illustrated embodiment, the base 22 and the cover 24 have generally symmetric shapes.

In certain embodiments, the base 22 and the cover 24 include one or more perforations or holes 30 which extend through the base 22 and the cover 24, respectively. While the one or more holes 30 are illustrated as having a circular shape, other shapes can also be employed. Further, the sizes of the one or more holes 30 may vary in regions of the cage 20. The holes 30 allow the transfer of air from inside the cage 20 to outside the cage 20 and vice versa. The transfer of air may decrease the likelihood that humidity in the air within the cage 20 will reach a level adverse to patient safety. In certain embodiments, the one or more holes 30 are disposed in the cage 20 so that a healthcare provider can visually determine the condition of the catheter assembly 10 without removing the cage 20.

Preferably, the one or more holes 30 are not disposed in the top surface of the cage 20 so as to prevent the patient from accessing a top of the catheter assembly 10 by extending a needle through the one or more holes 30. Of course if a cap 16 is present covering the lumen into the catheter assembly 10, the cap 16 may provide an adequate barrier to the needle entering the lumen in the catheter assembly 10.

In certain embodiments, the base 22 and/or cover 24 are manufactured from a clear material to allow the healthcare provider to visually determine the condition of the catheter assembly 10 even when the cage 20 is in the closed condition and/or does not include the one or more holes 30.

The cover 24 is releasably secured to the base 22 by one or more latch mechanisms, locks, or closures 32. In this preferred embodiment, the one or more latch mechanisms 32 encircle at least a portion of the cage 20 at a location midway between a top end and a bottom end of the cage 20. Of course the latch mechanism 32 could be disposed at other locations on the cage 20 as long as the latch mechanism 32 inhibits the patient from opening the cage 20 when in the closed condition. For example, a first lock 32 could be located at a side of the cage 20 while a second lock is located at the top end of the cage 20.

At least a portion of the latch mechanism 32 is disposed so that a healthcare provider can cause the latch mechanism 32 to fail (e.g. by cutting the lock mechanism) and open the cage 20. In certain embodiments, a portion of the latch mechanism 32 is accessible while another portion of the latch mechanism 32 is inaccessible when the cage 20 is in the closed condition. For example, a ligament portion of a strap of the latch mechanism 32 can be accessible while the remainder of the latch mechanism 32 is disposed and inaccessible inside the cage 20. In certain embodiments, a recess, hole, or opening in the cage 20 is sized so that the healthcare provider can access the internal ligament within the cage 20 to sever the ligament releasing the cover 24 from the base 22. In certain embodiments, the healthcare provider employs a scalpel, hemostat, medical scissors, pliers, or other cutting, twisting or crushing instrument to sever the ligament releasing the cover 24 from the base 22. In the illustrated embodiments, the latch mechanism 32 is disposed entirely outside the cage 20. Thus, the healthcare provider could employ medical scissors to cut the latch mechanism 32 at one or more locations around the perimeter of the latch mechanism 32 to release the cover 24.

In certain embodiments, the latch mechanisms 32 comprises at least one engagement structure disposed on the base 22 and one engagement structure disposed on the cover 24. The engagement structures are configured to engage together to secure the cover 24 to the base 22. In the illustrated embodiment, the base 22 comprises a first engagement structure in the form of an opening or passageway 36 while the cover 24 comprises a second engagement structure in the form of a strap 34. The passageway 36 is sized and shaped to allow the strap 34 to enter the passageway 36 but not retract from the passageway 36 once entered.

In alternative embodiments, the latch mechanism 32 is formed in the shape of one or more filaments with each filament having one or more protuberances. The one or more filaments could be disposed on the base 22 or the cover 24. The other of the base 22 or the cover 24 includes one or more receptacles configured to receive the filaments and the one or more protuberances when the cage 20 is in the closed condition. For example, in certain embodiments the base 22 includes four filaments disposed at the top, bottom, and sides of the base 22. The latch mechanism 32 of course can include other numbers of filaments in order to suit a specific application or size of cage 20.

A fixed proximal end of each filament is attached to the base 22 with a free distal end of the filament extending towards a receptacle on the cover 24. Between the fixed proximal end and the free distal end are at least one protuberance positioned therebetween. In certain embodiments, each filament includes a plurality of protuberances arranged in series between the distal end and the proximal end of the filament. The protuberances generally have identical barb-like shapes. In certain embodiments, each protuberance of the filament has a generally conical shape with a maximum diameter at a proximal end of the protuberance. The protuberances can take a variety of other shapes, such as for example, hollow conical shapes, arrow shapes, or transverse rib-like shapes. The proximal end of each protuberance, however, desirably has a diameter which is larger than the diameter of the filament. As such, the proximal end of each protuberance forms a flat surface that lies generally transverse to a longitudinal axis of the corresponding filament. The proximal end surface of some or all of the protuberances alternatively can slop or project toward the distal end of the filament.

The receptacles on the cover 24 include apertures. Each aperture advantageously has a conical or funnel-like shape to help guide the distal end of the filament through the aperture. The aperture tapers from a large diameter to a smaller diameter through the receptacle. The smaller diameter desirably is larger than the maximum diameter of the filament distal portion, but smaller than the maximum diameter of the protuberances on their proximal sides.

The receptacles positioned on the cover 24 are preferably arranged to cooperate with at least one filament. For example, each receptacle receives the distal end of the filament in a manner permitting the insertion of the filament into the receptacle, but inhibiting the retraction of the filament from the receptacle. For this purpose, the corresponding filament and receptacle include the protuberance and the aperture, respectively, that allows the filament distal end to be easily inserted into the receptacle in one direction with a first degree of force but prevents retraction of the filament distal end when a same degree of force is applied to the filament in the opposite direction. The degree of force required to retract the filament would result in breaking the filament or the receptacle of the cage 20. The cut or broken filaments or receptacles provide a visual indication to the healthcare provider that the cage 20 has been opened.

In alternative embodiments, the latch mechanism 32 is formed in the shape of prongs on one of the base 22 or the cover 24. The other of the base 22 or the cover 24 includes a lip or recess which engages with the prongs when the cage 20 is in the closed condition. To disengage the prongs, a portion of the prongs is cut or broken to thereby open the cage 20. The cut or broken prongs provide a visual indication to the healthcare provider that the cage 20 has been opened.

In another alternative embodiment, the latching mechanism 32 comprises a nub positioned along an edge of one of the base 22 and the cover 24 so as to engage a ledge of a catch on the other one of the base 22 and the cover 24. As with the prior embodiment, a portion of the nub or catch is cut or broken to open the cage 20. The cut or broken latching mechanism 32 provides a visual indication to the healthcare provider that the cage 20 has been opened.

In another alternative embodiment, the latching mechanism 32 comprises an adhesive material positioned between adjacent surfaces of the base 22 and the cover 24. As with the prior embodiment, a portion of the latching mechanism 32 such as the adhesive is cut or broken to open the cage 20. The cut or broken latching mechanism 32 provides a visual indication to the healthcare provider that the cage 20 has been opened. The adhesive can be any type of adhesive that when applied to the surfaces of the base 22 and the cover 24 binds them together and resists separation of the base 22 from the cover 24.

In the illustrated embodiment, the latch mechanism 32 includes one passageway 36 and one strap 34. The passageway 36 is configured to receive and engage with at least a portion of the strap 34, as will be described in further detail below. In the illustrated embodiment, the passageway 36 extends in a transverse direction adjacent to an edge of the base 22. In certain embodiments, the passageway 36 is angled slightly to facilitate the healthcare provider aligning the strap 34 with the passageway 36. The passageway 36 has a length sufficient to support the strap 34.

The illustrated strap 34 is a one-piece flexible plastic strap 34 coupled to the cover 24. The width of the strap 34 desirably is sufficiently long to provide stability to the cage 20 when secured about the cage 20. That is, the width of the strap 34 is sufficient to inhibit rotation of the cover 24 relative to the base 22. The strap 34 has an elongate shape which desirably has a sufficient length to circumscribe the cage 20 for embodiments where the strap 34 is a separate structure. The strap 34 may have a shorter length when the strap 34 is coupled to an edge of the cover 24 as is illustrated in FIG. 3. In the illustrated embodiment, the strap 34 need not circumscribe the entire cage 20 to reach the passageway 36. In certain embodiments, the strap 34 has a sufficient longitudinal length to enter the passageway 36 of the latch mechanism 32.

As will be apparent from the below description, several features of the cage 20 (e.g., the strap) desirably are flexible. The cage 20 is formed of a material which maintains its shape even if the cage 20 were squeezed or crushed by the patient. Exemplary materials include plastics and the like. Suitable rigid but flexible materials include, for example, but without limitation, plastics, polymers or composites such as polypropylene, polyethylene, polycarbonate, polyvinylchloride, acrylonitrile butadiene styrene, nylon, olefin, acrylic, polyester, as well as moldable silicon, thermoplastic urethane, thermoplastic elastomers, thermoset plastics and the like. The illustrated base 22 and cover 24 may be formed by injection molded using polyethylene or polypropylene material or nylon. However, other materials can be utilized, and the cage 20 can comprise a unitary base 22, cover 24, and latching mechanism 32.

The strap 34 comprises an elongated base portion 42 having a free end 44. The free end 44 of the strap 34 is configured for insertion into and engagement with the passageway 36. The illustrated embodiment includes one strap 34 and one passageway 36. The strap 34 at its proximal end is connected to the cover 24. The strap 34 includes one or more teeth 38 which are configured to engage corresponding structure of the passageway 36, as will be described in further detail below.

As can also be seen in FIG. 3, the strap 34 is disposed laterally of the cage 20, near the passageway 36 when the cage 20 is in the closed configuration. The illustrated strap 34 is cantilevered from a side of the cover 24 when the cage 20 is in an open condition. The strap 34 can be configured to flex with respect to the cover 24 of the cage 20 to which it is attached, so as to facilitate engagement and/or disengagement of the strap 34 with the passageway 36. The healthcare provider introduces the free end 44 of the base portion 42 into the passageway 36 so that the one or more teeth 38 on the strap 34 engage with the passageway 36 to lock the strap 34 in the closed condition.

The free end 44 of the base portion 42 may be tapered toward its extremity, which is rounded, so as to facilitate entry into the passageway 36 in the latch mechanism 32. The teeth 38 are provided on the base portion 42 near the free end 44 and facilitate gripping of the free end 44 by the healthcare provider and locking the free end 44 in the passageway 36 of the latch mechanism 32.

The passageway 36 can include one or more teeth, pawls or protrusions 40. The protrusions 40 cooperate with the teeth 38 of the strap 34 to slightly inhibit longitudinal movement of the strap 34 through the passageway 36 while preventing retracting of the strap 34 from the passageway 36. In the illustrated embodiment, the passageway 36 has one transversely extending tooth 40 on the same side as the teeth 38 on the free end 44 of the strap 34. The tooth 40 is adapted to cooperate with the teeth 38 on the base portion 42 of the strap 34 so as to retain the base portion 42 within the passageway 36 of the latch mechanism 32. During use, the healthcare provider simply pushes the strap 34 in a lateral direction passing the teeth 38 by the one or more protrusions 40 until the cover 24 is secured to the base 22. By such a configuration, the protrusions 40 and the teeth 38 cooperate to prevent removal of the strap 34 from the passageway 36.

Of course in certain embodiments, the engagement structures of the latch mechanism 32 need not be disposed on the base 22 and the cover 24. In the illustrated embodiment, the base 22, the latching mechanism 32 including the strap 34, and the cover 24 are formed together as the cage 20. This can be accomplished in any of a variety of ways well known to those skilled in the art. For instance, the entire cage 20 can be injection molded in order to reduce fabrication costs. Alternatively, the strap 34 is separately formed and assembled with the base 22 and the cover 24 to comprise the cage 20. In such an embodiment, the base 22 and the cover 24 can be formed as a unitary structure by being injection molded together. The cage 20 is assembled by feeding the strap 34 through the opening 36 of the unitary base 22 and cover 24.

For embodiments where the strap 34 is separately formed from the base 22 and the cover 24, the latch mechanism 32 could be disposed on an end of the strap 34. In such an embodiment, the longitudinal length of the strap 34 is selected to accommodate a portion of the latch mechanism 32 similar to the passageway 36. Thus, the passageway 36 may be located on an end of the strap 34 or on the base 22. The passageway 36 operates to secure at least an end of the strap 34 as described more fully below. The strap 34 also desirably is of a dimension which provides for easy manipulation. For example, the strap's size easily accommodates the grasp of a healthcare provider.

The strap 34 is selectively attached to the cage 20 when the healthcare provider desires to secure the base 22 to the cover 24. For embodiments where the strap 34 is a separate structure from the base 22 and the cover 24, the separate strap 34 could be similar to a bag-tie which would allow the bag-tie to self-secure once wrapped around the cage 20. In such an embodiment, the cage 20 can include a narrow waist to prevent the patient from sliding the bag-tie off the cage 20 after the bag-tie has been secured around the cage 20. For example, outer cross-sections of the cage 20 could taper towards the middle of the cage 20. In certain embodiments, a channel, a belt-loop type of structure, or groove is formed on an outer surface of the cage 20 to prevent the bag-tie from being slid off the cage 20 even when the cage 20 has an outer cross-section that does not taper along its longitudinal length.

As most clearly shown in FIG. 3, the passageway 36 extends through the base 22 and is configured to receive the strap 34. Once inserted into the passageway 36, the strap 34 extends from both ends of the passageway 36. In certain embodiments, the passageway 36 has a width that is less than a length of the base 22. The passageway 36 may have multiple portions aligned in the lateral direction and forming a single path for the strap 34 about the cage 20. An opening or window extending through a wall of the passageway 36 may separate the passageway portions. The opening or window may advantageously ease access to a ligament of the strap 34. The portion of the strap 34 that is exposed through the window can be cut or broken by the healthcare provider to release the cover 24 from the base 22.

The passageway 36 may taper in width along at least a portion of its length. For example, the tapering or wide-mouth shapes of the passageway 36 eliminate an edge or surface over which the strap 34 could bind. The passageway 36 may further curve parallel with the strap 34 when the strap 34 enters the passageway 36 so as to smoothly guide the strap 34 as the strap 34 exits the passageway 36.

Tapering the ends of the passageway 36 advantageously eases insertion of the strap 34 into the passageway while maintaining a close fit between inner walls of the passageway 36 and the strap 34 between the tapering ends. Alternatively, a cross-section of the passageway 36 may substantially exceed the cross-section of the strap 34. When inserted, the strap 34 is fed through the passageway 36 until the inserted end extends from the other side of the passageway 36.

In addition to the advantages apparent from the above description of the preferred embodiment and the various alternative embodiments, the cage 20 has the advantage of inhibiting access to the medical article 10 or at least visually indicating to the healthcare provider if the patient accessed the medical article 10 by damaging the latching mechanism 32 of the cage 20. Unlike when the cage 10 is installed, only damaging the cage 20 will open the cage 20. If a patient forces the cage 20 open, the resulting condition of the cage 20 will be noticed by the healthcare provider. The necessity of damaging the cage 20 to access the contents of the cage 20 substantially inhibits the patient from accessing the medical article 10 or at least makes the healthcare provider aware if the patient does access the medical article 10. In this way, the healthcare provider may take additional measures with the patient to ensure that the patient does not repeatedly access the medical article 10 to self-medicate. As a result of this construction, many patients will be denied access to lumens through which the patient could self-medicate.

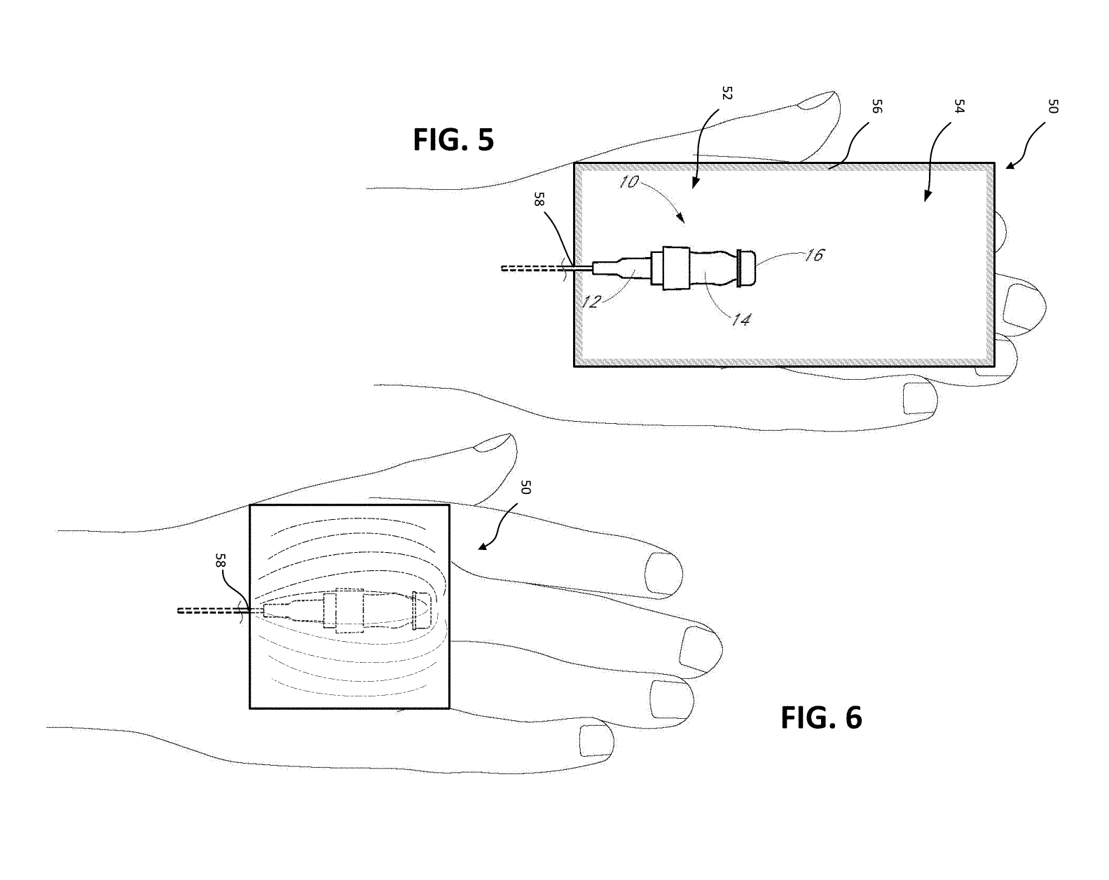

In some embodiments, the medical safety device can be a sheath 50 that is configured to receive at least a portion of the catheter assembly 10 or other medical article, as shown in FIGS. 5 and 6. The sheath 50 can be folded, wrapped, or otherwise positioned over or around the catheter assembly 10 to enclose at least a portion of the catheter assembly 10 to inhibit access to the lumen (e.g., through the port). The sheath 50 can be formed and positioned around the catheter assembly 10 such that the patient cannot access the lumen of the catheter assembly 10 without tampering with the sheath 50. The sheath 50 can be sized and configured to extend along and enclose any portion and/or length of the catheter assembly 10. For example, the sheath 50 can be configured to be positioned over and enclose the cap 16 on the end of the needless valve 14. In some embodiments, the sheath 50 can be positioned over the cap 16 and extend along and enclose various portions of the valve 14. The sheath 50 can enclose each of the cap 16, the needless valve 14, and the catheter hub 12. For example, the sheath 50 can extend and enclose the entire catheter assembly 10. In some embodiments, the sheath 50 can be positioned over and enclose a portion of the catheter shaft. As discussed herein, the sheath 50 can be positioned on any of the medical articles listed above to inhibit the patient from accessing the medical article (e.g., to self-medicate). One skilled in the art can also find additional applications for the devices and systems disclosed herein. Thus, the description of the sheath 50 in connection with a catheter assembly 10 is merely exemplary of one possible application of the sheath 50.

Unless otherwise noted, the components of the sheath 50 described herein are the same as or generally similar to the components of any other embodiments discussed herein. While the sheath 50 can incorporate features not discussed in other medical articles discussed herein, it will be understood that the features described with reference to the sheath 50 can be used with any of the embodiments described and/or contemplated herein. Any one of the medical articles disclosed herein can be modified to include any one of the features of the sheath 50. It will also be understood that any feature, structure, material, step, or component of any embodiment described and/or illustrated herein can be used with or instead of any other feature, structure, material, step, or component of any embodiment of the sheath 50.

As illustrated in FIGS. 5 and 6, a sheath 50 can be made of a single unitary body that can be folded, wrapped, or otherwise positioned over or around the catheter assembly 10 to inhibit access to the lumen. For example, a first portion 52 of the sheath 50, in some instances, can be positioned beneath at least a portion of the catheter assembly 10 (e.g., the catheter hub 12), and a second portion 54 of the sheath 50 can be folded over the catheter assembly 10 to be adhered to the first portion 52 of the sheath 50 to enclose at least a portion of the catheter assembly 10 and inhibit access to the lumen. For example, as illustrated in FIG. 6, the sheath 50 can cover the catheter assembly 10 to inhibit access to the lumen of the valve 14. The sheath 50 can be folded in the lateral direction (as shown in FIG. 6) and/or the longitudinal direction to cover at least a portion of the catheter assembly 10. The sheath 50 can be configured such that the first portion 52 of the sheath 50 is positioned below the catheter assembly 10 and the second portion 54 of the sheath 50 is positioned above the catheter assembly 10 relative to the patient's skin. The sheath 50 can be configured to adhere to itself when positioned to enclose at least a portion of the catheter assembly 10, such that the patient cannot pull apart the sheath 50 without producing a visual indication of tampering. In some embodiments, the first portion 52 and the second portion 54 of the sheath 50 can adhere to each other and/or the catheter assembly 10 along an engagement portion 56 of the sheath 50. The engagement portion 56 can comprise any adhesive and/or locking mechanism disclosed herein. In some embodiments, the engagement portion 56 can extend along an entire perimeter of the sheath 50. The sheath 50, in some instances, can comprise a non-adhesive portion that defines an opening 58 when the sheath 50 is in a closed configuration. The opening 58 can be sized and configured to selectively permit at least a portion of the catheter assembly 10 to pass through. An inner diameter of the opening 58 can be sufficiently large to permit at least a portion of the catheter assembly 10 (e.g., the smaller outer diameter of the catheter hub 12 or shaft) to reside within the opening 58. However, in some embodiments, the inner diameter of the opening 58 cannot be large enough to permit the remainder of the catheter assembly 10 to pass through the opening 58. As such, the catheter assembly 10 generally is too large to fit through the opening 58 and can only be removed upon destruction of the sheath 50. In some instances, however, the opening 58 can be large enough to permit the catheter assembly 10 to fit through the opening 58 with the sheath being maintained on the catheter assembly by another mechanism (e.g., by a zip tie or similar latching structure or by adhering directly to a portion of the catheter assembly).

In some embodiments, the sheath 50 when in the closed configuration (as illustrated in FIG. 6) can be formed by wrapping, enveloping, or crimping the sheath 50 around or over at least a portion of the catheter assembly 10 to cover at least an end portion of the catheter assembly 10 and to act as a barrier to access the needleless valve 14.

The sheath 50 can be configured to engage and/or adhere to a portion of the sheath 50 itself, as discussed herein, and/or any portion of the catheter hub 12. As shown in FIG. 5, the catheter assembly 10 can comprise various portions of differing diameters. For example, the catheter hub 12 can have a smaller diameter than the needleless valve 14 and/or the cap 16. Similar to the cage 20 as described herein, the sheath 50 can be secured to the catheter hub 12 to prevent the patient from slipping or removing the sheath 50 off the catheter assembly 10 if the catheter shaft were pulled by the patient.

In some embodiments, the sheath 50 can be adhered to at least one of the needleless valve 14 and/or the cover 16. The catheter shaft can have a smaller diameter than other components of the catheter assembly 10, such as the catheter hub 12, the needless valve 14, and the cap 16. The sheath 50 can be adhered to the catheter shaft such that the patient cannot remove or slip the sheath 50 over the catheter assembly 10 without producing a visual indication of tampering.

The sheath 50 can be secured to the outer diameter of the catheter assembly 10. In other embodiments, the sheath 50 can be secured to the surfaces that face perpendicular to the longitudinal axis of the catheter assembly 10.

The sheath 50 can be square, rectangular, oval, or any other suitable size and/or shape to enclose a medical article or a portion of a medical article (e.g., an access into a portion of a medical article). The sheath 50 can be substantially flat for ease of manufacturing and assembly. The sheath 50 can be rectangular or square for ease of manufacturing. In some embodiments, the sheath 50 can also be formed by securing a preformed or shaped structure like a cap or enclose at least a portion of the catheter assembly 10 to inhibit access to the lumen of the catheter assembly 10.

In some embodiments, the sheath 50 can be made of two or more separate body portions that can be configured to enclose various portions of the catheter assembly 10. The two or more body portions can be shaped and positioned to engage and/or attach to at least one of the catheter assembly 10 and another of the two or more body portions. For example, the sheath 50 can be formed by securing two or more body portions that can be positioned to encase the catheter assembly 10 between the two or more body portion to inhibit access to the lumen of the catheter assembly 10.

The sheath 50 can be made of a variety of flexible or semi-rigid materials such as polyester film or sheet, plastic sheet or film, or PET (polyethylene terephthalate). For example, the sheath 50 can comprise Mylar® polyester film. The sheath 50 can be formed by folding a plastic sheet or film over the catheter assembly 10, as described herein. In some embodiments, the sheath 50 can comprise a material configured to provide an indication a healthcare practitioner if a patient has tampered with the sheath 50. For example, the sheath 50 can be made of a material that, if punctured with a needle or other vascular access device, would show unauthorized access to the lumen and could not be mended or repaired to conceal the unauthorized access. As such, the puncture hole in the sheath 50 would indicate to the healthcare practitioner that the patient has tampered with the sheath 50 and accessed or attempted to access the needleless valve 14.

The sheath 50 can be shaped or formed to the particular contours of the catheter assembly 10 or the medical article. For example, if the medical article is Y-shaped as described above, the sheath 50 can be formed by two Y-shaped sheets that are adhered or secured together around or to the perimeter of the Y-shaped medical article.

The visual indication of tampering, as described herein, can be the absence of the sheath 50, absence of the portion of the sheath 50, puncturing or penetration of the sheath 50, repositioning of the sheath 50, lack of adherence of the sheath 50 to the catheter assembly 10, and/or any additional evidence of re-adherence or replacement of the sheath 50.

The sheath 50 can be made of plastic sheet or film, which can provide ease of manufacturing as well as low manufacturing costs. The plastic sheet or film can be produced in large quantities through a variety of methods such as fabricating, molding, thermoforming, extrusion, casting, converting, and machining including cutting, stamping, and/or carving. The sheath 50 can be secured to the catheter assembly 10 using a variety of adhesives. In some embodiments, the sheath 50 can be made of an adhesive material or made of material with adhesive that has been pre-applied. The adhesives can be structural or pressure-sensitive. Adhesives can include epoxy, cyanoacrylate, acrylic based light cured, or solvent bonding. The adhesive may be heat activated, temperature activated, pressure activated, chemically activated, hardened by UV radiation, or bonded by other appropriate means. Such adhesives can be applied to a portion of or to all of one side of the sheath. For example, an adhesive layer can extend about a portion of or the entire perimeter of the sheath. In addition or as an alternative, adhesive can be positioned on the plastic sheet or film to adhere to some or all of the catheter assembly 10.

The sheath 50 can also be secured to the catheter assembly 10 using any other locking mechanisms disclosed herein to prevent the patient from removing the sheath 50. The sheath 50 can, in some instances, be secured to the catheter assembly 10. For example, the sheath 50 can provide a seal at any location along the catheter assembly 10 (e.g., the end of the needless valve 14) to inhibit access of the lumen of the catheter assembly 10. In some embodiments, the sheath 50 can be secured to the catheter assembly 10 irreversibly, such that the sheath 50 cannot be removed and/or reused without indications of tampering. The sheath 50, in some instances, cannot be impenetrable or completely prevent access to the lumen of the catheter assembly 10. The sheath 50 can be penetrated or punctured with a needle such that the patient can access the lumen of the catheter assembly 10. The sheath 50 can provide a barrier to access the catheter assembly 10 that provides indication of tampering. The sheath 50 can be made of a material that cannot be easily removed or replaced.

The size and shape of the sheath 50 can be appropriately sized to match or to approximate the size and shape of the catheter assembly 10 or a portion of the catheter assembly 10. For example, in accordance with one exemplary form, the length of the sheath 50 when assembled can be approximately 1 inch to approximately 4 inches. The width or diameter of the sheath 50 can be approximately 0.5 inches to approximately 1 inch. The film or sheet can be approximately 2 mm thick to approximately 3 mm thick. In other embodiments, the sheath 50 can be applied to other varieties of medical devices or other types of catheter assemblies of different shapes and dimensions. The sheath 50 can be appropriately sized to accommodate alternate sized catheter assemblies or medical articles.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above.

What is claimed is:

1. A medical safety device configured to irreversibly lock about at least a portion of a medical article, the device comprising:
a base;
a cover configured to be positioned on the base to form a cage, the cage being sized to enclose the medical article; and
a latching mechanism configured to irreversibly lock the cover to the base so as to inhibit access to a lumen in the medical article.

2. The medical safety device of claim 1, wherein the medical article is a catheter assembly, in particular the portion of the medical article is a valve of the catheter assembly, further in particular wherein the portion of the medical article is a top surface of the valve.

3. The medical safety device of claim 2, wherein the catheter assembly comprises one or more abutment surfaces, and wherein the cage comprises one or more contact surfaces, the one or more abutment surfaces being configured to cooperate with the one or more contact surfaces to inhibit movement of the catheter assembly when in the cage and the cover is locked to the base.

4. The medical safety device of any of claim 1, wherein the medical article includes a side port, and wherein the cage encloses at least the side port, in particular wherein the cage comprises one or more holes, further in particular wherein the latching mechanism comprises a strap, further in particular wherein the latching mechanism comprises a passageway configured to receive at least a portion of the strap.

5. The medical safety device of claim 1, wherein the latching mechanism comprises a filament and a receptacle, in particular wherein the filament comprises one or more protuberances, further in particular wherein the receptacle comprises an aperture, the aperture and the one or more protuberances being sized to allow the one or more protuberances to pass through the aperture in only one direction.

6. The medical safety device of claim 1, wherein the latching mechanism comprises an adhesive, in particular further comprising a hinge between the base and the cover.

7. The medical safety device of claim 1, wherein at least one of the base and the cover comprises a channel, the channel being configured to receive an edge of the other one of the base and the cover when the cover is positioned on the base.

8. The medical safety device of claim 1, wherein the cage has an elongated oval shape with a circular cross-section.

9. A medical safety device configured to inhibit access to at least a portion of a medical article, the device comprising:
a base and a cover, the cover being movable relative to the base between a first position and a second position, the base and the cover being configured to receive the portion of the medical article when in the first position and to enclose the portion of the medical article when in the second position; and
a latching mechanism configured to secure the cover to the base so as to inhibit the portion of the medical article from being removed from the base and the cover when the base and the cover are in the second position.

10. The medical safety device of claim 9, wherein the base and the cover form a receptacle when in the second position, in particular wherein the portion of the medical article is a top of a valve.

11. The medical safety device of claim 9, wherein the portion of the medical article is a side port, and wherein the base and the cover enclose at least the side port when in the second position.

12. The medical safety device of claim 9, wherein at least a portion of the latching mechanism is accessible for a healthcare provider to cut or break the latching mechanism releasing the cover from the base, in particular wherein the latching mechanism is a latch on a strap which secures the strap to itself, further in particular wherein the latching mechanism comprises a one-way, self-locking, non-releasable passageway, further in particular comprising a hinge between the base and the cover.

13. A method for inhibiting access to at least a portion of a medical article, the method comprising:
providing a cage comprising a first portion and a second portion, the second portion being movable relative to the first portion;
disposing the portion of the medical article in the cage;
positioning the first portion relative to the second portion to enclose the portion of the medical article in the cage; and
latching the first portion to the second portion so as to inhibit at least the portion of the medical article from being removed from the cage.

14. The method of claim 13, wherein the latch mechanism comprises a ligament portion, and further comprising severing the ligament portion with a medical instrument to open the cage.

15. The method of claim 13, wherein the latch mechanism comprises an adhesive, and further comprising cutting the adhesive with a medical instrument to open the cage.

16. A medical safety device configured to irreversibly lock about at least a portion of a medical article, the device comprising:
a sheath comprising a first body portion and a second body portion, the first body portion configured to be positioned on the second body portion to form an enclosed space, the enclosed space being sized to enclose at least a portion of the medical article; and
a fastening mechanism configured to irreversibly lock the first body portion to the second body portion so as to inhibit access to a lumen in the medical article.

17. The medical safety device of claim 16, wherein the medical article is a catheter assembly and wherein the portion of the medical article is a valve of the catheter assembly.

18. The medical safety device of any of claim 16, wherein the medical article includes a side port.

19. The medical safety device of claim 1, wherein the fastening mechanism comprises an adhesive.

20. The medical safety device of claim 1, wherein the sheath further comprises opening configured to selectively receive at least a portion of the catheter assembly.
